(19) United States
(12) Patent Application Publication
(10) Pub. No.: US 2016/0221892 A1
(43) Pub. Date: Aug. 4, 2016

(54) SYSTEM AND METHOD FOR EFFICIENTLY USING EXCESS ELECTRICAL ENERGY

(71) Applicant: EVONIK DEGUSSA GMBH, Essen (DE)

(72) Inventors: Georg Markowz, Alzenau (DE); Thomas Wenzel, Frankfurt (DE); Steffen Heinecke, Dortmund (DE); Ingrid Lunt-Rieg, Bad Homburg (DE); Surya Liauw, Bogor, West Java (ID)

(73) Assignee: Evonik Degussa GmbH, Essen (DE)

(21) Appl. No.: 15/021,158

(22) PCT Filed: Sep. 5, 2014
(86) PCT No.: PCT/EP2014/068890

§ 371 Date: Mar. 10, 2016

(30) Foreign Application Priority Data
Sep. 11, 2013 (DE) 10 2013 218 175.2

(51) Int. Cl. C07C 2/80; B01J 19/00; C07C 2/82; B01J 7/00

(52) U.S. Cl. CPC C07C 2/80; B01J 7/00; B01J 19/0046; C07C 2/82; B01J 2219/00452; B01J 2219/00585; B01J 2219/00716; B01J 2219/0072

(57) ABSTRACT

A plant having a first device for the preparation of ethyne by partial oxidation of at least one hydrocarbon, generating a first ethyne-containing product gas stream, having a second device for the electrothermal preparation of ethyne, generating a second ethyne-containing product gas stream, and having a separating device for separating ethyne from a gas stream, to which both the first and the second product gas streams are fed, can make efficient use of excess electrical energy by operating the device for the electrothermal preparation of ethyne with excess electrical energy.

The present invention relates to a plant and to a method for the efficient utilization of excess electrical energy, in which the electrical energy is utilized for the preparation of ethyne.

The use of renewable energies such as wind energy and solar energy is gaining ever increasing importance for power generation. Electric energy is typically brought to a large number of consumers via long-range, super regional power supply grids coupled over national borders, referred to as power grids for short. Since electric energy cannot be stored to a significant extent in the power grid itself, the electric power fed into the power grid has to be matched to the consumer-side power requirement, known as the load. The load is known to fluctuate in a time-dependent manner, in particular depending on the time of day, day of the week or even time of the year. For a stable and reliable power supply, continuous equality of power generation and power uptake is necessary. Any short-term deviations which occur are equalized by means of positive or negative control energy or control power. In the case of renewable power generation facilities, there is the difficulty that in the case of particular types, e.g. wind energy and solar energy, energy generation does not occur at every point in time and cannot be controlled in a definite manner but is subject to fluctuations according to the time of day and weather conditions, which fluctuations are foreseeable to only a limited extent and generally do not match the energy demand at the particular time.

The difference between power output from fluctuating renewable energies and the actual consumption is usually provided by other power stations such as gas, coal and nuclear power plants. With increasing expansion of fluctuating renewable energies and the proportion of power supply represented by them, ever larger deviations between their power output and actual consumption have to be equalized. Thus, at the present time gas power plants and increasingly also hard coal power plants are operated at part load or shut down entirely in order to compensate the fluctuations. Since this variable mode of operation of the power plants is associated with considerable additional costs, the development of alternative measures has been examined for some time.

One approach is, in the case of an excess of electric energy, to utilize excess electric energy for the electrothermal preparation of ethyne, as an alternative to or in addition to changing the power output of a power plant. An example of this was the ethyne plant of the Hills chemical works, which had 19 arc reactors in parallel and in which the number of arc reactors in operation was varied in dependence on the supply of electrical energy. While arc reactors for the electrothermal production of ethyne can be turned on and off quickly, an efficient and economical removal of ethyne from the product gas stream obtained in the electrothermal preparation of ethyne requires a highly constant product gas stream. The ethyne plant at the Hills chemical works, with an ethyne capacity of 120 000 t/a, therefore comprised buffer reservoirs for the product gas stream with a total volume of 350 000 m³. The construction and operation of buffer reservoirs of such size, however, is technically costly and complicated, and involves safety risks.

There is therefore a need for plants and methods with which excess electrical energy can be utilized via the preparation of ethyne, and which do not have the disadvantages of the method described above.

The invention provides a plant for the efficient utilization of excess electrical energy, comprising:
a first device for the preparation of ethyne by partial oxidation of at least one hydrocarbon, generating a first ethyne-containing product gas stream,
a second device for the electrothermal preparation of ethyne, generating a second ethyne-containing product gas stream, and
a separating device for separating ethyne from a gas stream, to which both the first and the second product gas streams are fed.

The invention additionally provides a method for the efficient utilization of excess electrical energy, where, in a plant according to the invention, the device for the electrothermal preparation of ethyne is operated with excess electrical energy.

The invention further provides a method for providing control energy for an electricity network, in which, in a plant according to the invention, both the first device for the preparation of ethyne by partial oxidation of at least one hydrocarbon and the second device for the electrothermal preparation of ethyne are operated under part load; for the provision of control energy, the output of the second device for the electrothermal preparation of ethyne is altered; and with a control device, the output of the first device for the preparation of ethyne by partial oxidation of at least one hydrocarbon is adapted in such a way that the total amount of ethyne separated in the separating device is maintained within a specified range.

The plant of the invention comprises a first device for the preparation of ethyne by partial oxidation of at least one hydrocarbon, generating a first ethyne-containing product gas stream. This first device may comprise one or more apparatuses in which ethyne is generated by partial oxidation. If the first device comprises a plurality of apparatuses for the generation of ethyne, they are preferably arranged in parallel and can be operated independently of one another. The use of a plurality of units arranged in parallel allows stepwise alteration of the production of ethyne while maintaining optimal operating conditions in the individual units by switching on and switching off individual units and avoids efficiency losses due to partial load operation.

As the first device in the plant of the invention, it is possible to use all of the devices known from the prior art for the preparation of ethyne by partial oxidation, examples being the Sachsse-Bartholomé process and the BASF submerged flame process devices, known from Ullman's Encyclopedia of Industrial Chemistry, 5th Edition, Vol. A1, pages 107-110 and 113-114, or the Montecatini process device known from GB 1,000,480. The first device for the preparation of ethyne by partial oxidation preferably comprises at least one burner fed with a mixture of at least one hydrocarbon and oxygen.

In addition to the first device for the preparation of ethyne by partial oxidation, the plant of the invention also comprises a second device for the electrothermal preparation of ethyne, generating a second ethyne-containing product gas stream. The second device may comprise one or more apparatuses in which ethyne is generated electrothermally. If the second device comprises a plurality of apparatuses for the generation of ethyne, they are preferably arranged in parallel and can be operated independently of one another. The use of a plurality of units arranged in parallel allows stepwise alteration of the production of ethyne while maintaining optimal operating conditions in the individual units by switching on and switching off individual units and avoids efficiency losses due to partial load operation.

In an electrothermal preparation of ethyne, ethyne is prepared in an endothermic reaction from hydrocarbons or carbon and the heat required for carrying out the reaction is generated by electric power. Preference is given to using gaseous or vaporized hydrocarbons, particularly preferably aliphatic hydrocarbons. Methane, ethane, propane and butanes, in particular methane, are particularly suitable. Suitable devices for the electrothermal preparation of ethyne are known from the prior art, as for example from Ullmann's Encyclopedia of Industrial Chemistry, 5th Edition, Vol. A1, pages 115-122, from DE 1 900 644 A1 and from EP 0 133 982 A2.

The device for the electrothermal preparation of ethyne preferably comprises an electric arc reactor. The electrothermal preparation of ethyne can be carried out in a single-stage process in which at least one hydrocarbon is passed through the electric arc with a gas stream. As an alternative, the electrothermal preparation of ethyne can be carried out in a two-stage process in which hydrogen is passed through the electric arc and at least one hydrocarbon is fed downstream of the electric arc into the hydrogen plasma generated in the electric arc. The device for the electrothermal preparation of ethyne preferably comprises a plurality of electric arc reactors which are arranged in parallel and can be operated independently of one another.

The plant of the invention further comprises a separating device for separating ethyne from a gas stream, the separating device being supplied both with the first product gas stream from the first device for the preparation of ethyne by partial oxidation of at least one hydrocarbon, and with the second product gas stream from the second device for the electrothermal preparation of ethyne. The separating device for separating ethyne preferably comprises a compressor, an absorption column operated under pressure, and a desorption column operated under a lower pressure than the absorption column. Water or suitable solvents, such as, for example, N-methylpyrrolidone, dimethylformamide or methanol, can be used for the selective absorption of ethyne. Suitable separating devices for separating ethyne are known from the prior art, as for example from Ullmann's Encyclopedia of Industrial Chemistry, 5th Edition, Vol. A1, pages 110-112.

In a preferred embodiment, the plant of the invention further comprises a control device which matches the generation of ethyne in the first device and in the second device to one another in such a way that the total amount of ethyne separated in the separating device is maintained within a specified range. The total amount of ethyne separated in the separating device is preferably held substantially constant. For this purpose the control device preferably comprises measuring devices for determining the mass flow rate or volume flow rate of the first and second product gas streams, analytical devices for determining the ethyne content of the first and second product gas streams, and devices for altering the output of the first device for the preparation of ethyne by partial oxidation and of the second device for the electrothermal preparation of ethyne.

The first and the second devices for the preparation of ethyne preferably each comprise a device for the rapid cooling (quenching) of product gas stream. The gas streams obtained after these separate devices for rapid cooling are fed to the separating device for separating ethyne. These product gas streams are preferably cooled to temperatures of less than 250° C. The rapid cooling may be accomplished using a direct quenching method such as, for example, the introduction of hydrocarbons and/or water, or an indirect quenching method, such as, for example, rapid cooling in a heat exchanger with generation of steam. Direct quenching and indirect quenching may also be combined with one another. In a first embodiment, the gas mixture leaving the reaction zone is quenched only with water. This embodiment features relatively low capital costs. In a preferred embodiment, the gas mixture leaving the reaction zone is mixed with a hydrocarbon-containing gas or with a hydrocarbon-containing liquid, with at least part of the hydrocarbons being cracked endothermically. Depending on the process regime, a more or less broad product spectrum is produced, for example fractions of ethane, propane, ethene and other lower hydrocarbons in addition to ethyne, hydrogen and possibly carbon monoxide. As a result, the heat produced can be passed on to a substantially greater extent to a further use, such as the endothermic cracking of hydrocarbons. Suitable devices for quenching the product gas stream are known from the prior art, as for example from Ullmann's Encyclopedia of Industrial Chemistry, 5th Edition, Vol. A1, pages 108-110 and 116-118.

With particular preference, the first and the second devices for the preparation of ethyne each comprise a device for the rapid cooling of product gas stream and a downstream device for the removal of soot. The gas streams obtained after the devices for the removal of soot are fed to the separating device for separating ethyne. For the removal of soot, it is possible to use all of the devices employed for this purpose in known methods for the preparation of ethyne, examples being cyclones, scrubbers or electrostatic precipitators. Suitable devices are known, for example from Ullmann's Encyclopedia of Industrial Chemistry, 5th Edition, Vol. A1, pages 108-110 and 118. The use of separate devices for the removal of soot for the first and the second devices for the preparation of ethyne permits better utilization of the soot produced in the method; for example, the soot obtained in the device for the electrothermal preparation of ethyne can be utilized as carbon black pigment, and the soot obtained in the device for the preparation of ethyne by partial oxidation can be used as a fuel.

The plant of the invention preferably further comprises, between the device for the electrothermal preparation of ethyne and the separating device for separating ethyne, a buffer reservoir for a product gas stream of the device for the electrothermal preparation of ethyne. Alternatively or additionally, the plant of the invention may further also comprise, between the device for the preparation of ethyne by partial oxidation and the separating device for separating ethyne, a buffer reservoir for a product gas stream of the device for the preparation of ethyne by partial oxidation. Particularly suitable buffer reservoirs are gasometers. A buffer reservoir allows the plant of the invention to be operated such that in the event of a change in the output of the second device, the change in the generation of ethyne in the first device takes place with a time offset or at a different speed, and a resultant greater or smaller generation of product gas is balanced by the introduction of product gas into the buffer reservoir or the withdrawal of product gas from the buffer reservoir.

The method of the invention for the efficient utilization of excess electrical energy is carried out in a plant of the invention, and the device for the electrothermal preparation of ethyne is operated with excess electrical energy. The excess electrical energy may come from an electricity generator located adjacent to the plant of the invention, for example a neighbouring power plant, a neighbouring wind generator or a neighbouring photovoltaic plant. The excess electrical energy is preferably taken from an electricity network. With particular preference, excess electrical energy is taken from an electricity network in the form of negative control energy, in order to compensate an excess in the electricity introduced into the network relative to the electricity withdrawn at the moment. The excess electrical energy used for the method of the invention is preferably energy generated from wind energy or solar energy.

In the method of the invention for the efficient utilization of excess electrical energy, the device for the electrothermal preparation of ethyne is preferably operated in dependence on the supply of excess electrical energy. The device for the electrothermal preparation of ethyne may for this purpose be turned on or off selectively, in dependence, for example, on the current electricity price at an electricity exchange. Alternatively, the first device may also be operated with variable load in such a way that its electricity consumption corresponds to a current excess of electrical energy.

In a preferred embodiment, the method of the invention for the efficient utilization of excess electrical energy is carried out in a plant of the invention which comprises a buffer reservoir for a product gas stream, and the control device is operated such that in the event of a change in the generation of ethyne in the second device, in dependence on the supply of excess electrical energy, the generation of ethyne in the first device is changed more slowly than the generation of ethyne in the second device, and the resultant temporarily greater or smaller overall generation of product gas is balanced by the introduction of product gas into the buffer reservoir or by the withdrawal of product gas from the buffer reservoir. This buffer reservoir may selectively be positioned downstream of the first device or of the second device. It is also possible for both devices to have a downstream buffer reservoir. With this embodiment, the generation of ethyne in the second device can be changed more quickly, in dependence on the supply of excess electrical energy, and restrictions on the speed of load changes, which are inherent to the process of devices for the preparation of ethyne by partial oxidation, can be overcome.

In a further preferred embodiment, a gas stream, which has been depleted of ethyne in the separating device for separating ethyne, is recycled to the separating device with the second ethyne-containing product gas stream. The amount of the recycled gas stream in this case is adjusted such that the fraction of ethyne, based on the total amount of gas streams fed to the separating device, remains substantially constant. With particular preference, the recycled gas stream is fed to the separating device together with the first and second product gas streams. Inherent to the process, the first product gas stream from the device for the preparation of ethyne by partial oxidation has a significant fraction of carbon monoxide. Furthermore, it generally has a substantially lower ethyne content than the second product gas stream from the device for the electrothermal preparation of ethyne. Recycling of an ethyne-depleted gas stream allows for balancing the difference in the ethyne content of the two product gas streams and prevents that a change in the load distribution between the first and second ethyne-generating devices negatively affects the operation of the separating device due to the difference in the composition of the product gas streams from the two devices.

The method of the invention for providing control energy for an electricity network is carried out in a plant of the invention which comprises a control device which matches the generation of ethyne in the first device and in the second device to one another in such a way that the total amount of ethyne separated in the separating device is maintained within a specified range. In this method, both the first device for the preparation of ethyne by partial oxidation of at least one hydrocarbon and the second device for the electrothermal preparation of ethyne are operated under part load. For the provision of control energy, the output of the second device for the electrothermal preparation of ethyne is altered; and with the control device, the output of the first device for the preparation of ethyne by partial oxidation of at least one hydrocarbon is adapted in such a way that the total amount of ethyne separated in the separating device is maintained within a specified range.

If less electrical energy than is currently being consumed is introduced into the electricity network from which electricity is taken to operate the device for the electrothermal preparation of ethyne, it is possible with this method to provide positive control energy, by reducing the output of the device for the electrothermal preparation of ethyne in line with the demand for control energy, and, correspondingly, raising the output of the device for the preparation of ethyne by partial oxidation of at least one hydrocarbon, by way of the control device. If, in contrast, more electrical energy is being fed in to the electricity network than is being currently consumed, this method can be used to provide negative control energy, by raising the output of the device for the electrothermal preparation of ethyne in accordance with the demand for control energy, and, correspondingly, reducing the output of the device for the preparation of ethyne by partial oxidation of at least one hydrocarbon, by way of the control device.

1-14. (canceled)

15. A plant for the efficient utilization of excess electrical energy, comprising:
a) a first device for the preparation of ethyne by partial oxidation of at least one hydrocarbon, generating a first ethyne-containing product gas stream;
b) a second device for the electrothermal preparation of ethyne, generating a second ethyne-containing product gas stream; and
c) a separating device for separating ethyne from a gas stream, to which both the first and the second product gas streams are fed.

16. The plant of claim 15, further comprising a control device which matches the generation of ethyne in the first device and in the second device to one another, thereby maintaining the total amount of ethyne separated in the separating device within a specified range.

17. The plant of claim 15, wherein the first device for the preparation of ethyne by partial oxidation comprises at least one burner fed with a mixture of oxygen and at least one hydrocarbon.

18. The plant of claim 15, wherein the second device for the electrothermal preparation of ethyne comprises at least one arc reactor.

19. The plant of claim 15, wherein the first and the second device for the preparation of ethyne each comprise a device for the rapid cooling of product gas stream, and the gas streams obtained after these devices for rapid cooling are fed to the separating device for the removal of ethyne.

20. The plant of claim 15, wherein the first and the second devices for the preparation of ethyne each comprise a device for the rapid cooling of product gas stream and a downstream device for the removal of soot, and the gas streams obtained after the devices for the removal of soot are fed to the separating device for separating ethyne.

21. The plant of claim 15, further comprising a buffer reservoir for a product gas stream of the device for the electrothermal preparation of ethyne between the device for the electrothermal preparation of ethyne and the separating device for separating ethyne.

22. The plant of claim 15, further comprising a buffer reservoir for a product gas stream of the device for the preparation of ethyne by partial oxidation between the device for the preparation of ethyne by partial oxidation and the separating device for separating ethyne.

23. A method for the efficient utilization of excess electrical energy, wherein in a plant according to claim 15, the device for the electrothermal preparation of ethyne is operated with excess electrical energy.

24. The method of claim 23, wherein the excess electrical energy is taken from an electricity network.

25. The method of claim 23, wherein the excess electrical energy is generated from wind energy or solar energy.

26. The method of claim 23, wherein the device for the electrothermal preparation of ethyne is operated in dependence on the supply of excess electrical energy.

27. The method of claim 23, wherein a gas stream depleted of ethyne in the separating device for separating ethyne is recycled to the separating device with the second ethyne-containing product gas stream, and the amount of the recycled gas stream is adjusted such that the fraction of ethyne, based on the total amount of gas streams fed to the separating device, remains substantially constant.

28. A method for providing control energy for an electricity network, wherein in a plant according to claim 15, both the first device for the preparation of ethyne by partial oxidation of at least one hydrocarbon and the second device for the electrothermal preparation of ethyne are operated under part load; for the provision of control energy, the output of the second device for the electrothermal preparation of ethyne is altered; and with the control device, the output of the first device for the preparation of ethyne by partial oxidation of at least one hydrocarbon is adapted in such a way that the total amount of ethyne separated in the separating device is maintained within a specified range.

29. The method of claim 28, wherein, in said plant, the first device for the preparation of ethyne by partial oxidation comprises at least one burner fed with a mixture of oxygen and at least one hydrocarbon.

30. The method of claim 28, wherein, in said plant, the second device for the electrothermal preparation of ethyne comprises at least one arc reactor.

31. The method of claim 28, wherein, in said plant, the first and the second device for the preparation of ethyne each comprise a device for the rapid cooling of product gas stream, and the gas streams obtained after these devices for rapid cooling are fed to the separating device for the removal of ethyne.

32. The method of claim 28, wherein, in said plant, the first and the second devices for the preparation of ethyne each comprise a device for the rapid cooling of product gas stream and a downstream device for the removal of soot, and the gas streams obtained after the devices for the removal of soot are fed to the separating device for separating ethyne.

33. The method of claim 28, wherein said plant further comprises a buffer reservoir for a product gas stream of the device for the electrothermal preparation of ethyne between the device for the electrothermal preparation of ethyne and the separating device for separating ethyne.

34. The method of claim 28, wherein said plant further comprises a buffer reservoir for a product gas stream of the device for the preparation of ethyne by partial oxidation between the device for the preparation of ethyne by partial oxidation and the separating device for separating ethyne
